(19) United States
(12) Patent Application Publication
(10) Pub. No.: US 2007/0197879 A1
(43) Pub. Date: Aug. 23, 2007

(54) Diagnostic tool

(76) Inventor: Alexandra Martz, Brussels (BE)

Correspondence Address: JACOBSON HOLMAN PLLC, 400 SEVENTH STREET N.W., SUITE 600, WASHINGTON, DC, 20004, US

(21) Appl. No.: 11/646,573

(22) Filed: Dec. 28, 2006

(30) Foreign Application Priority Data
Dec. 29, 2005 (EP) 05113069.8

(51) Int. Cl. A61B 5/00

(52) U.S. Cl. 600/300

(57) ABSTRACT

Diagnostic tool comprising a sheet representing a human or animal body, which body is divided into a set of zones, a first identifier being attributed to each zone, which tool also comprises, for each diagnostic to be applied, a set of indicators formed by a first indicator reproducing the first identifier attributed to the first of said zones where a person in charge of making the diagnostic must apply at least one finger of one of his or her hands and a sequence of second indicators reproducing the first identifiers attributed to the successive points attributed in zones thus identified where said person must successively apply at least one finger of his or her other hand.

The invention relates to a diagnostic tool which can be used to diagnose an illness or dysfunction of the human or animal body. The invention relates also to a therapeutic tool.

Such a diagnostic tool is generally used by physicians, kinesitherapists or more generally health professionals in the exercise of their profession. It allows the diagnostic of an illness or a dysfunction of the human or animal body, as well as the physician to determine the therapeutic treatment to be applied.

A disadvantage of the known diagnostic tools is that they only take into account fractions of the human or animal body and not the functioning of the human body in its whole.

The invention has as objective the realisation of a diagnostic tool allowing linking the illnesses and other dysfunctions of the human or animal body as well as the symptoms which are associated with topographies of the whole of the body.

For this purpose, a diagnostic tool according to the invention is characterised in that it comprises a sheet representing a human or animal body, which body is divided into a set of zones, a first identifier being attributed to each zone, which tool also comprises for each diagnostic to be applied, a set of indicators formed by a first indicator reproducing the first identifier attributed to the first of said zones where a person in charge of making the diagnostic has to apply at least one finger of one of his or her hands and a sequence of second indicators reproducing the first identifiers attributed to the successive points attributed in zones so identified where said person has to successively apply at least one finger of his or her other hand.

Thanks to the first indicator and the sequence of second indicators, the person in charge of making the diagnostic can, according to the diagnostic to be applied, perfectly determine the places in the body where he or she must first apply at least one finger of one hand and thereafter a finger of the other hand. In this manner, by applying successively fingers on well determined locations of the body, pairs of points on the body will be successively activated, which should cause an activation of the nervous, circulatory and/or osseous systems. This activation will then permit the person carrying out the diagnostic to establish how the body reacts, which will then allow to establish the diagnostic. The various first identifiers thus provide a topography of the body, which allows to link various points of the body with each other and to act on the whole of the body functions.

It must be noted that the patent application US 2004/0230256 describes an electronic, electrical and electromagnetic device for improving and stimulating health. The device described in this patent application is however not a diagnostic tool, but in fact a therapeutic tool. The known device utilizes a set of points spread over the body. These points are however not zones, as is the case in the present invention and there is thus not a division of the body, since the points indicate solelyaline to be applied to the body, but do not divide the body in zones. Certainly, the known device recognizes the concept of a first indicator, but not that of the identifier. Indeed, to have an identifier as in the present invention a division in zones is imperatively necessary. Even if the description refers to two locations on the body, there is no link between those locations, as is the case in the present invention.

A first preferred embodiment of a diagnostic tool according to the invention is characterized in that each of said zones of said set of zones is subdivided in a predetermined number of sub-zones, a second identifier being attributed to each sub-zone, said first and second indicators reproducing also the second identifiers. The subdivision in sub-zones allows a more precise topography of the body which also allows to realise the diagnostic in a systematic manner by an easier localisation for the person carrying out the diagnostic.

A second preferred embodiment of a diagnostic tool according to the invention is characterised in that said first identifiers comprise numbers, which are attributed by increasing order starting by a lower part of said body. The use of numbers by increasing order allows a logical and systematic approach to the division of the body.

The invention will now be described in more detail with the support of the drawings illustrating embodiments of a diagnostic tool according to the invention.

In the drawings:

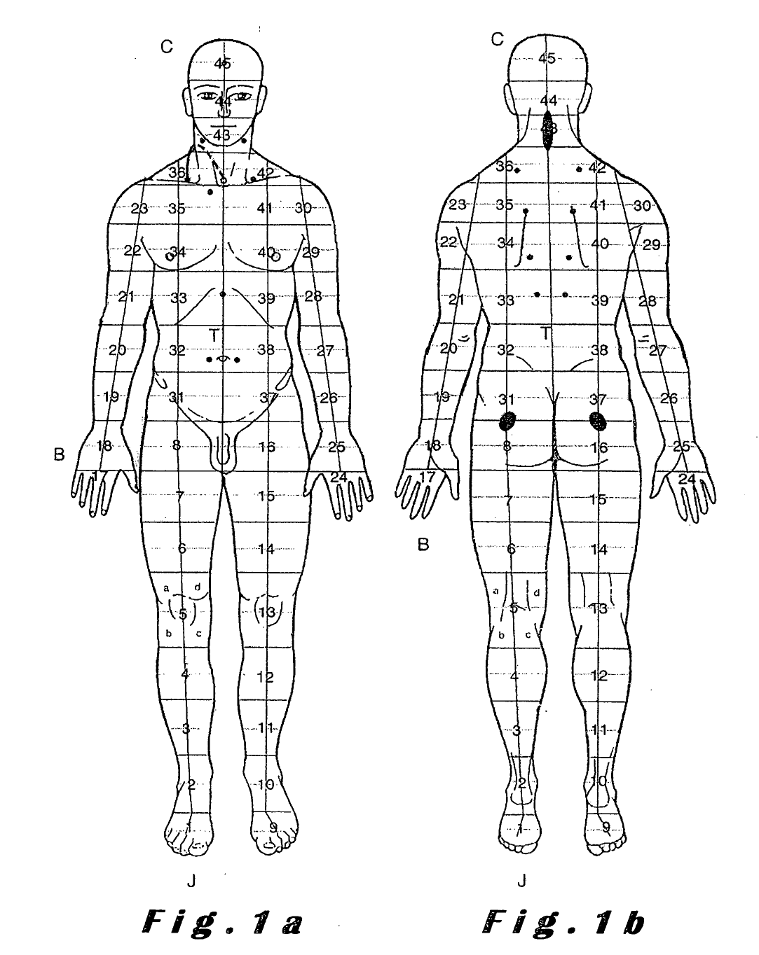

FIGS. 1a and b show a first embodiment of a diagnostic tool according to the invention and which is suitable for diagnosing allergies; and

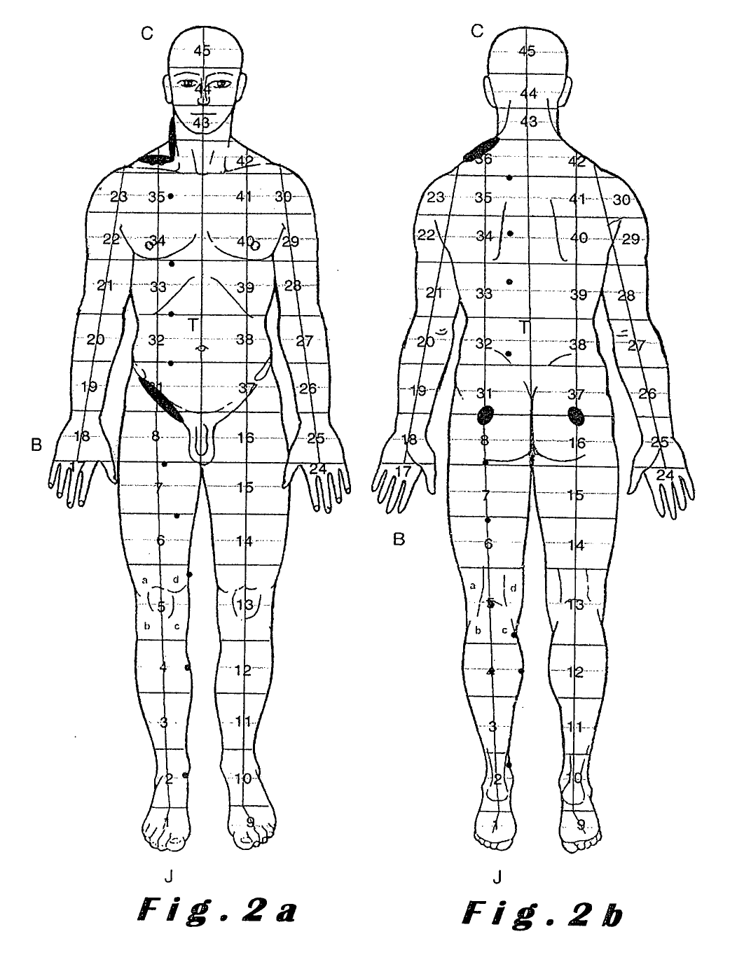

FIGS. 2a and b show a second embodiment of a diagnostic tool according to the invention and which is suitable for diagnosing blood circulation problems.

In the drawings, a same reference has been attributed to the same or an analogous element.

The diagnostic tool according to the invention and shown in FIG. 1 comprises a sheet representing a human body, in this case the ventral (1a) and dorsal (1b) sides of a man. In the human body reproduced in the sheet the arms are in an anti-anatomical position, since the thumbs point towards the legs. The objective of this manner of representing the human body is to force the person, who will apply the diagnostic, to impose to the person for whom the diagnostic is to be established to put himself in this anti-anatomical position. Indeed, in this anti-anatomical position a relaxation of the body and particularly of the nerves and the tendons is obtained, which permits to make a more reliable diagnostic. Of course, the invention is not limited to that only representation of the ventral and dorsal sides of a man and can also comprise a representation of the right or left profiles of a man, as well as representations of the dorsal, ventral sides of a woman and a representation of her profiles. It is also not limited to be used to apply a diagnostic on an adult person, but also to children or babies. Since the invention applies also to a diagnostic tool for an animal body, it can also comprise representations of the body of various animals.

The sheet is formed by a sheet of paper, cardboard or a synthetic or natural material on which the representation of the body is printed. The sheet can also be formed by a computer screen or any other support allowing the display of information. The body is divided in a set of zones. A first identifier is attributed to each zone. In the example illustrated in FIG. 1, the first identifiers comprise numbers attributed by increasing order and starting at a lower part of the body, in this case the right foot. This numbering, which begins at the lower part of the body, corresponds to a diagnostic logic where the left hand is preferably always in a higher position with respect to the right hand. Of course, other forms of the first identifiers other than numbers can be used, such as, for instance, letters or symbols. In place of starting the numbering at the foot, it is of course equally possible to start it at any other location of the body, such as, for instance, a hand or the head. It must however be noted that the sheet usually indicates only a half-body and that the points represented on the support are only represented unilaterally. A numbering symmetrical with respect to a median axis m of the body could also be applied. Moreover, as the drawings show, a division with respect to a median axis is also applied with respect to the legs, arms and head. For what concerns the division in zones of the legs, the knee has been considered as central 5 or 13 and three zones are located in each case starting from this central zone. For what concerns the division in zones of the arms, the elbow forms an axis and three zones are located in each case starting from this axis.

In the example illustrated in FIG. 1, the body is divided in a set of substantially rectangular zones. This is of course a preferential embodiment, since the division in rectangles can be simply and efficiently applied. However, other divisions than that in rectangles can also be realised, such as, for instance, divisions in triangles or trapeziums. The size of each zone can either be equal or different in surface. It is however preferable to take account of the body's own structure in applying the division in zones so as to give a coherent structure to the zone. Thus, for instance, the brain will form a single zone.

To make the localisation of the zones easier and quicker, the first identifiers comprise also uppercase letters. Thus, for example, the following letters are attributed:

J: leg

T: trunk

B: arm

C: skull

Each zone of the set of zones is subdivided in a predetermined number of sub-zones. In the example illustrated in FIG. 1, each rectangular zone is subdivided in four rectangles in order to refine the division of the body and to make the determination of a topographic point on the body more precise. To each sub-zone is attributed a second identifier. In the example illustrated in FIG. 1, the second indicators are formed by the letters a, b, c and d, each of which being attributed to one of the sub-zones. The attribution is similar for each sub-zone and follows a counter-clockwise direction starting from the top left sub-rectangle.

For each diagnostic to be applied, the diagnostic tool according to the invention comprises also a set of indicators formed by a first indicator reproducing the first identifier attributed to the first one of said zones where a person in charge of making the diagnostic in question must apply at least one finger of one of his hands. Thus, for example, in the sheet illustrated in FIG. 1 and table 1, reproducing a diagnostic for verifying the existence of an allergy and the therapy to be applied, the first indicator is formed by T36c (reproduced on the ventral side)+C43bc reproduced on the dorsal side of the human body. This means that the person having to apply the diagnostic will first have to apply at least one finger of one of his or her hands, for example the thumb of his or her left hand, on the zone T36, i.e. in zone 36 of the trunk, more particularly in the lower sub-rectangle (c). At the same time, he or she will apply at least another finger of the same hand in zone C43 of the dorsal side of the skull (as indicated in the nomenclature), more particularly in the bottom left sub-rectangle (b) and bottom right sub-rectangle (c). In order to ease the task of said person even further, the first indicator is, preferably, represented by a predetermined symbol reproduced on the topographic representation illustrated on the drawings. That symbol is e.g. formed by a rectangle or another geometric shape. The set of indicators comprises also a sequence of second indicators reproducing the first indicators attributed to the successive points in the zones thus identified where said person should successively apply at least one finger of his other hand. In the allergy example illustrated in FIG. 1, this second indicator is formed by T33d+T39a and indicates the point to apply by one of the fingers of the right hand. It must be noted that the sequence of the second indicator must be applied by the person carrying out the diagnostic. Of course, this second indicator can also be indicated by a colour on the sheet, preferably the same used for the first indicator.

It must be noted that the person carrying out the diagnostic applies so to say a very light pressure or touch on the body to be diagnosed. The time during which at least one finger is applied is generally well under a minute.

Using a different colour for each diagnostic to be applied, it is possible to use one single plate to diagnose several different illnesses.

Using the set of indicators, the person that has to carry out the diagnostic can activate each time a couple of points on the body and generate so an activation of the osseous, circulatory and/or nervous systems with a predominance of one or the other, or even a shared complementarity. Thus, applying firstly at least one finger of one hand on the point indicated by the first indicator and thereafter at least one finger of the other hand on successively each of the points indicated by the second indicator, said person is going to induce a physiological reaction in the body on which the diagnostic has to be applied. This physiological reaction can, for example, translate into an electrical charge between those points and create between those points a potential in an electrical field. Thus an electrical current will possibly flow between those points. Said person will feel this current of which the parameters, such as for example the intensity or polarity, will have changed and will indicate him the reaction of the body on which the diagnostic is applied. Of course this reaction of the body will be completely determined by the body's functioning. The physiological reaction can also translate into a chemical reaction whose effects will also be felt by the person carrying out the diagnostic.

The sheet illustrated on FIG. 2 as well as table 2 reproduce a diagnostic for verifying the existence of a blood circulation problem and the therapy to be applied. In this diagnostic, the first indicator is formed by the first indicator T31abc+J8d. This means that the person having to apply the diagnostic will first have to place at least one finger of one of his or her hands, for example the middle finger of his or her left hand, or the palm of his or her hand in the zone T31, that is, in zone 31 of the trunk, more particularly in the sub-rectangles a, b and c. At the same time he or she will apply another finger of this same hand in zone J8d of the leg, more particularly in the top right sub-rectangle (d) of the leg, where he will watch that the palm of his hand will also cover this latter sub-zone.

In the blood circulation example illustrated in FIG. 2, the second indicator is formed by J2d, J4cd, J5d, J6d, J7d and indicates the points to apply successively by the right hand and so on, as indicated in the nomenclature reproduced hereunder.

The diagnostic tool according to the invention allows thus to link the illnesses as well as the symptoms which are associated with precise topographies of the body. The tool allows not only diagnosing but also treating the body, it thus has a preventive and curative aspect. Indeed, when the points indicated by the whole set of indicators have been activated, it will also be possible to stimulate the thus induced electrical current or chemical reaction, which will allow the body to restart functioning as a whole and regain by its own means a balance, which would have been disturbed.

Acting upon the bones, the blood circulation system and/or the nervous system, it is directly or indirectly acted upon the endocrine, immune, cardiac, musculoskeletal, lymphatic, cellular, tissue, physiological as well as on the digestive, respiratory and neurovegetative systems, and thus in a more global manner on the various systems constituting the human body. The act of activating electrical currents can provoke a membrane depolarisation between the two stimulated points. This membrane depolarisation is transmitted to the brain, which will then activate the mechanisms intervening in the treatment of the body dysfunction. The activated points can also play a role of chemical catalyser which will cause an enzymatic, thus more generally chemo-physiological activation, which, by a chain effect will transmit the necessary information about the repair of the failing system.

Illustratively, some examples of indicator sets will be reproduced hereunder. Within the diagnostic points reproduced hereunder, only the essential points are reproduced and this for reasons of clarity.

### Diagnostic Points

Female Genital System

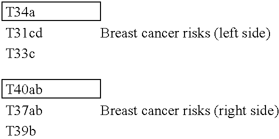

Male Genital System

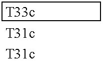

Prostate problems, incontinence, erectile trouble

Digestion

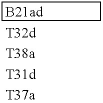

Back problems

Digestive trouble: swelling, pain . . .

Risk of cancer in serious cases (liver, pancreas)

Allergies

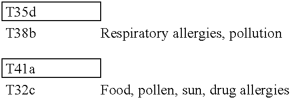

Circulatory System

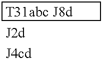

Circulatory trouble, at risk of phlebitis

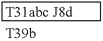

Heart risks with strong probability of family antecedents

Cerebrovascular accident risks

Fibromyalgia

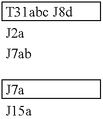

Bone Density for All Bones

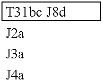

Low bone density

Infection

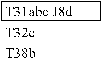

Dental, or skin infections, insect bite . . .

Anxiety

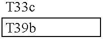

Patients subject to anguish, anxiety crises, low morale, phobias

TABLE 1
ALLERGIES

VENTRAL SIDE:

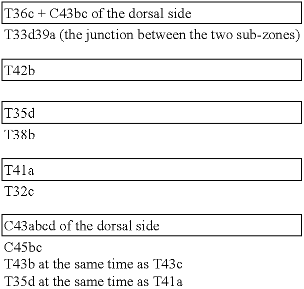

DORSAL SIDE:

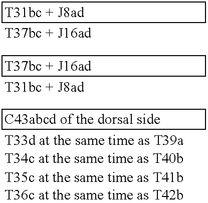

TABLE 2
BLOOD AND CIRCULATION

VENTRAL SIDE:

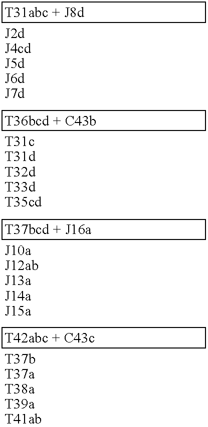

DORSAL SIDE:

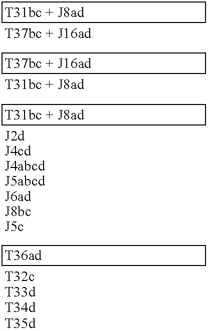

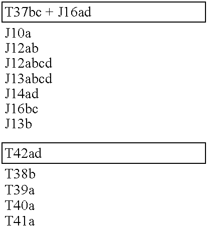

The tool according to the invention can not only be used for diagnostic purposes, but also for therapeutic purposes. Take the case of a person suffering from leg pain and having trouble to stand upright. In such cases, there is a strong possibility to believe that this person has precedents of phlebitis and varixes in his or her family. The objective is to verify if this person has effectively circulatory problems. The person who is going to carry out the diagnostic is thus going to use the sheet (FIG. 2a) and follow the indications provided in the nomenclature indicated in table 2. Thus he or she will place his or her hand on T31abc+J8d and his or her right hand successively on J2d and J4cd. To confirm his or her diagnostic he or she can place his or her right hand on J5d, J6d and J7d.

The reactivity of these points confirms at first the diagnostic. That these points be reactive means a feeling by the patient, such as for example an electrical current, a tingling feeling, heat, a tickle, a more or less important feeling of pressure, or even pain when the person applying the diagnostic is carrying out a light pressure or even a simple touch. This feeling should disappear as soon as the left hand is taken away. To confirm his or her own diagnostic, the therapist can go on more rapidly over the other points listed in the nomenclature corresponding to the FIG. 2a and table 2.

Having carried out the diagnostic, the person will carry out the therapy and will apply the complete sequence or part of the sequence of the points reproduced in the table 2 and represented partially in FIG. 2a and 2b. It must be noted that in most of these cases the same sequence of points to be applied in the therapeutic tool will be found as that used in the diagnostic tool.

1. Diagnostic tool characterised in that it comprises a sheet representing a human or animal body, which body is divided into a set of zones, a first identifier being attributed to each zone, which tool also comprises, for each diagnostic to be applied, a set of indicators formed by a first indicator reproducing the first identifier attributed to the first of said zones where a person in charge of making the diagnostic must apply at least one finger of one of his or her hands and a sequence of second indicators reproducing the first identifiers attributed to the successive points attributed in zones thus identified where said person must successively apply at least one finger of his or her other hand.

2. Diagnostic tool according to claim 1, characterised in that each of said zones of said set of zones is subdivided in a predetermined number of sub-zones, a second identifier being attributed to each sub-zone, said first and second indicators reproducing also the second identifiers.

3. Diagnostic tool according to claim 1, characterised in that the first indicator is attributed to the left hand and the second indicators to the right hand of said person.

4. Diagnostic tool according to claim 1, characterised in that said first identifiers comprise numbers, which are attributed by increasing order starting by a lower part of the body.

5. Therapeutic tool characterised in that it comprises a sheet representing a human or animal body, which body is divided in a set of zones, a first identifier being attributed to each zone, which tool also comprises, for each therapy to be applied, a set of indicators formed by a first indicator reproducing the first identifier attributed to the first of said zones where a person in charge of applying the therapy must apply at least one finger of one of his or her hands and a sequence of second indicators reproducing the first identifiers attributed to the successive points attributed in zones thus identified where said person must successively apply at least one finger of his or her other hand.
